(19) United States
(12) Patent Application Publication
(10) Pub. No.: US 2011/0118839 A1
(43) Pub. Date: May 19, 2011

(54) BONE PLUG

(76) Inventor: Robert Nutter, Reno, NV (US)

(21) Appl. No.: 12/942,461

(22) Filed: Nov. 9, 2010

Related U.S. Application Data
(60) Provisional application No. 61/281,179, filed on Nov. 13, 2009.

(51) Int. Cl. A61F 2/28

(52) U.S. Cl. 623/16.11

(57) ABSTRACT

A device for insertion into an amputated bone and a method for inserting the device into an amputated bone are described. The device comprises a stem configured to be inserted into a cavity of the amputated bone. The device further comprises a head coupled to the stem. The head has a diameter greater than the diameter of the stem. The head further has a flat surface configured to interface with a severed surface of the amputated bone. The head and stem are fabricated from a moldable porous matrix material configured to promote bone growth within the matrix. A device for bridging a first amputated bone and a second amputated bone is also described.

### CROSS-REFERENCING

The present patent application claims the benefit of provisional patent application No. 61/281,179, filed Nov. 13, 2009.

### FIELD OF THE INVENTION

The present invention relates to a system and method for plugging a cavity in an amputated bone. More specifically, the invention relates to a compressible bone plug capable of rapid insertion into a bone cavity.

### BACKGROUND

The remaining bone within an amputated limb may cause discomfort when the bone applies pressure to the tissue between the bone and a prosthetic device. Various capping devices have been applied to the remaining bone to distribute the pressure along a surface area that is greater than that of the bone tip.

Many existing devices for capping a bone are fabricated from material subject to rejection by the body, such as metals. Metal devices may also limit the patient's ability to undergo certain diagnostic procedures, such as magnetic resonance imaging (MRI). Thus, there remains a need for a plug fabricated from a matrix with which the bone may become integrated.

Devices having rigid shapes may require the bone to be modified, such as by drilling, to accommodate the cap. The rigid shape of such devices also prevent the device from accommodating changes in bone structure occurring, e.g., due to growth in children and adolescents or due to osteoporosis in the elderly. Thus, as the bone changes, the patient may require multiple surgeries for replacement of the device. There is a resulting need for a plug that is moldable to allow custom fabrication and has sufficient flexibility to accommodate changes in the bone structure over time.

Existing capping devices typically require cement or sutures to fix the device in place relative to the bone. The surgical process required for inserting such devices may be long and complex. A need remains for a plug that is elastic, allowing the device to expand when in place, thereby lessening the complexity of the surgical procedure associated with insertion of the device.

There is also a need for a device that may be inserted by an emergency medical responder into an amputated limb to stabilize the limb and prevent entry of foreign matter and debris into the bone cavity while the patient is being transported from the scene of the accident to a medical facility.

### SUMMARY

A device for insertion into an amputated bone is described. The device comprises a stem configured to be inserted into a cavity of the amputated bone. The device further comprises a head coupled to the stem. The head has a diameter greater than the diameter of the stem. The head further has a flat surface configured to interface with a severed surface of the amputated bone. The head and stem are fabricated from a moldable porous matrix material configured to promote bone growth within the matrix.

In another embodiment, a device for bridging a first amputated bone and a second amputated bone is described. The device comprises a first stem configured to be inserted into a cavity of the first amputated bone and a second stem configured to be inserted into a cavity of the second amputated bone. A first head is coupled to the first stem. The first head has a diameter greater than the diameter of the first stem. The first head has a flat surface configured to interface with a severed surface of the first amputated bone. A second head is coupled to the second stem. The second head has a diameter greater than the diameter of the second stem. The second head has a flat surface configured to interface with a severed surface of the second amputated bone. The device further comprises a bridging plate coupled to the first head and the second head.

In yet another embodiment, a method for inserting a bone plug into an amputated bone is described. The method comprises molding a head and stem from a porous matrix material configured to promote bone growth within the matrix. The head and stem are molded such that the head has a diameter greater than the diameter of the stem, the head has a flat surface configured to interface with a severed surface of the amputated bone, and the head is coupled to the stem. The method further comprises inserting the stem into a cavity of the amputated bone.

### DRAWINGS

The present invention will be more fully understood by reference to the following drawings which are for illustrative, not limiting, purposes.

FIG. 1A shows an illustrative perspective view of a bone plug having a dome shaped head.

FIG. 1B shows an illustrative bottom view of a bone plug having a dome shaped head.

FIG. 2A shows an illustrative perspective view of a bone plug having a hollow stem.

FIG. 2B shows an illustrative bottom view of a bone plug having a hollow stem.

FIG. 3A shows an illustrative perspective view of a bone plug having a knob shaped head.

FIG. 3B shows an illustrative perspective view of a bone plug having a knob shaped head and hollow stem.

FIGS. 4A-4B show an illustrative bone plug having compression ridges oriented along the circumference of the stem.

FIG. 5 shows an illustrative bridging device for bridging two bone plugs placed in parallel amputated bones.

FIGS. 6A-6B show an illustrative bone plug having a conical stem.

FIG. 7 shows an illustrative flowchart for inserting a bone plug into an amputated bone.

### DESCRIPTION

Persons of ordinary skill in the art will realize that the following description is illustrative and not in any way limiting. Other embodiments of the claimed subject matter will readily suggest themselves to such skilled persons having the benefit of this disclosure. It shall be appreciated by those of ordinary skill in the art that the apparatus and systems described herein may vary as to configuration and as to details. Additionally, the methods may vary as to details, order of the actions, or other variations without departing from the illustrative method disclosed herein.

The bone plug comprises a stem that is inserted into the cavity of an amputated bone. The stem prevents foreign matter from entering the bone cavity. In some embodiments, the bone plug further comprises a head coupled to the stem. The head has a diameter exceeding the diameter of the stem. A flat surface of the head abuts the severed surface of the amputated bone. The head and stem may be fabricated from a matrix material configured to promote growth of new bone within the matrix. The head portion of the bone plug may thus distribute the weight a patient puts on the amputated bone, e.g., when a prosthetic device is attached to the amputated limb. Rather than being concentrated at the tip of the amputated bone, weight placed on the amputated limb is distributed over the area of the head portion of the plug, while bone growth into the stem and head anchor the plug to the bone.

The bone plug may be applied to any amputated bone having sufficient diameter to receive a plug, including but not limited to the femur, tibia, fibula, humerus, radius, ulna and bones of the fingers.

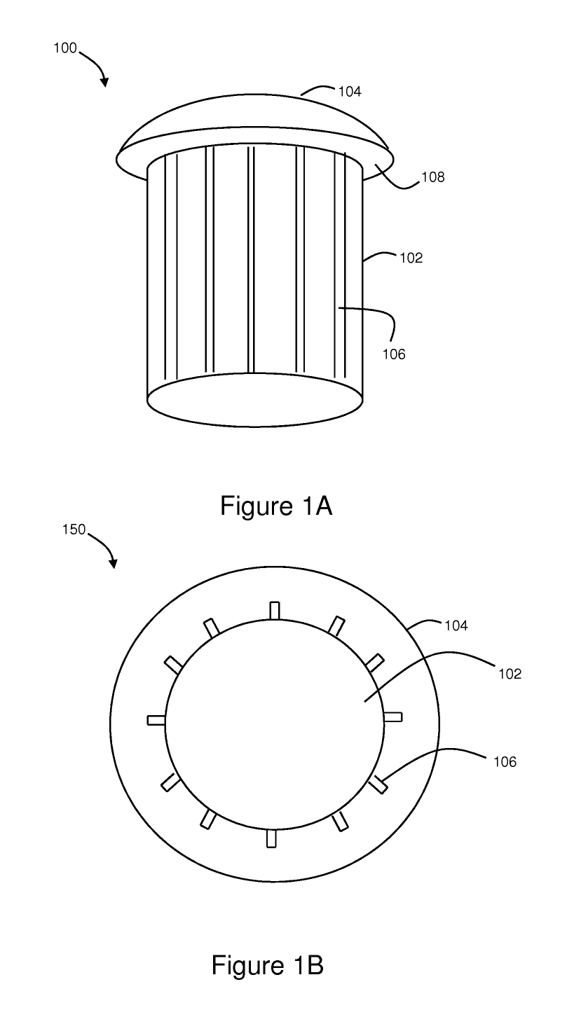

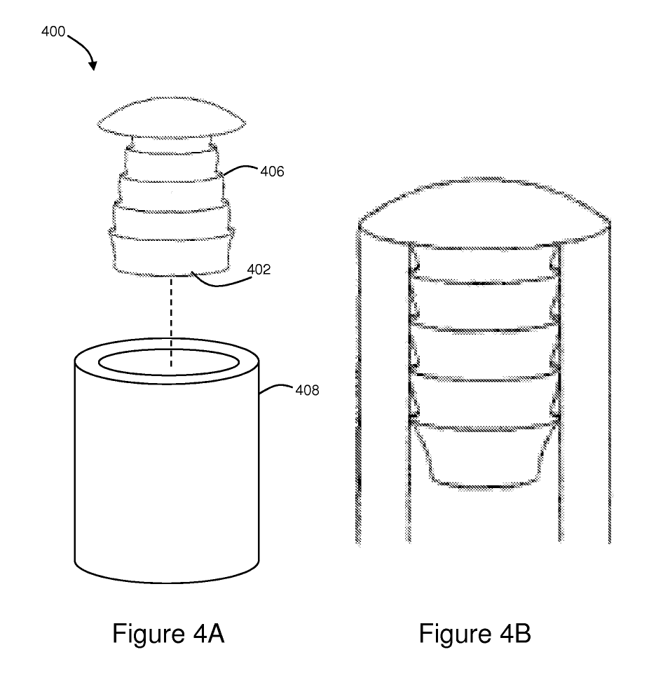

Referring to FIG. 1A, there is shown an illustrative perspective view 100 of a bone plug according to a first embodiment. Stem 102 of the bone plug is coupled to head 104. The head has a dome shape with a flat surface 108 that contacts the severed portion of the amputated bone and a round surface that contacts the tissue folded over the bone of the amputated limb. Compression ridges 106 are coupled to the exterior of the stem. In the embodiment shown in FIG. 1A, the compression ridges are a series of long, narrow ridges oriented along the length of stem 102. In an alternative embodiment, the compression ridges are oriented around the circumference of the stem, as shown in FIG. 4. Stem 102 is inserted into the bone cavity until the flat surface 108 of head 104 is flush with the severed surface of the amputated bone. The compression ridges 106 exert outward pressure on the interior surface of the bone cavity to anchor the stem in place within the bone cavity.

FIG. 1B shows an illustrative bottom view 150 of the bone plug according to a first embodiment. In FIG. 1B, the compression ridges 106 can be seen extending radially from stem 102.

The matrix material used to fabricate the bone plug is a porous material that provides sufficient open space to promote bone growth within the matrix. The matrix material may comprise a synthetic bone substitute such as tricalcium phosphate (beta TCP), biphasic ceramic made of hydroxyapatite (e.g., 75%) and tricalcium phosphate (e.g., 25%), or other orthophosphates. Alternatively, the matrix material may comprise bone material from the amputee's body or from other donors. Preferably, the matrix material is moldable to create a plug that is custom fitted for the bone cavity of the amputated limb. The matrix may be elastic to allow the plug and the compression ridges to be compressed for insertion into the bone and subsequently return to the molded shape after insertion. In some embodiments, the matrix is a putty-like substance that can be fitted to the amputated bone during surgery. The matrix may contain silicone or other moldable material to provide a putty-like substance.

In some embodiments, a tissue-adhesive material is applied to the rounded surface of the head to promote adhesion between the plug and the tissue. The tissue-adhesive material is a biologically compatible material having a higher rate of attachment to soft tissues than that of the bone matrix material.

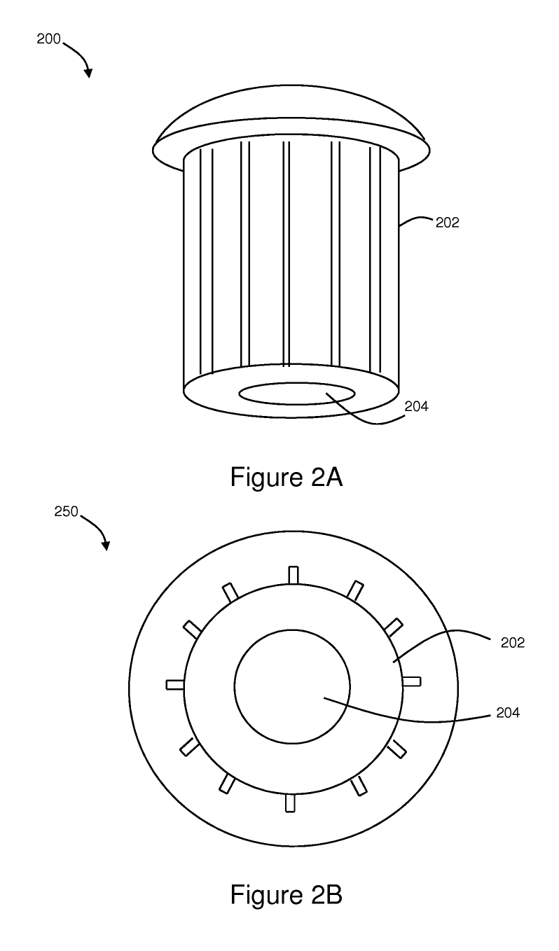

Referring now to FIG. 2A, there is shown an illustrative perspective view 200 of a bone plug according to a second embodiment. In the second embodiment, stem 202 of the bone plug comprises a shaft 204 that renders the stem hollow. FIG. 2B shows an illustrative bottom view 250 of the bone plug according to a second embodiment. In FIG. 2B, shaft 204 is shown within hollow stem 202. The shaft in stem 202 allows the stem to be radially compressed to aid in insertion of the plug into the bone cavity. The stem may subsequently expand within the bone cavity to anchor the plug within the cavity.

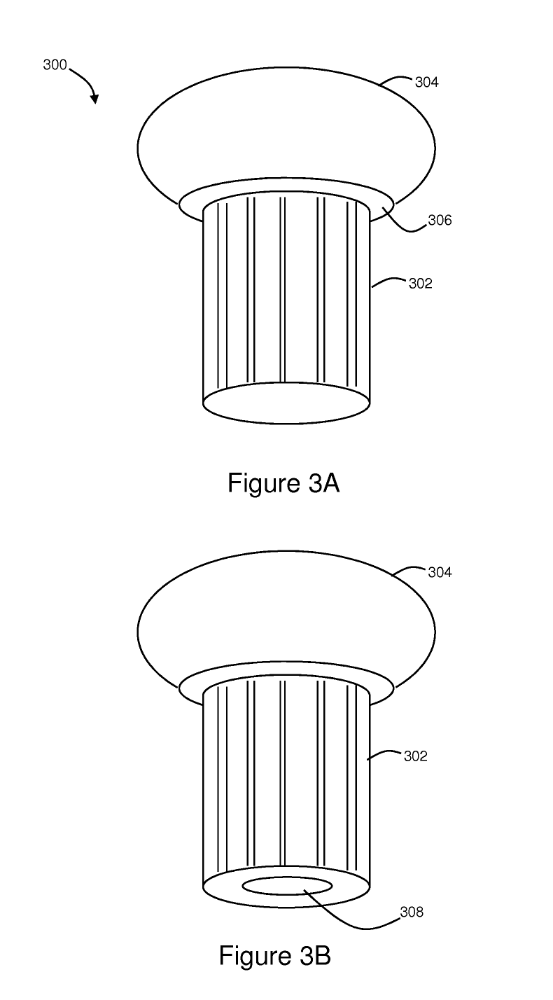

Referring now to FIG. 3A, there is shown an illustrative perspective view 300 of a bone plug according to a third embodiment. In the third embodiment, stem 302 is coupled to a head 304 having a flat surface 306 that is coupled to stem 302 and contacts the severed portion of the amputated bone. Head 304 has a knob-like surface that contacts the tissue folded over the bone of the amputated limb. Use of a knob-like head as shown in FIG. 3A in lieu of the dome shaped heads shown in FIGS. 1-2 is desirable when additional shock absorption is required. The larger volume of the head provides more shock absorption when a great deal of weight is applied to the amputated limb, for example, if the bone plug is used in a femur. Additionally, the greater surface area of the head results in greater diffusion of the force applied to the limb. FIG. 3B shows an illustrative perspective view 250 of the bone plug according to a fourth embodiment. In FIG. 3B, the bone plug has a knob-like head 304 and stem 302 is shown comprising a shaft 308.

Referring now to FIG. 4A, there is shown an illustrative bone plug 400 according to a fifth embodiment. In FIG. 4A, compression ridges 406 are oriented around the circumference of stem 402 of the bone plug. The bone plug in 4A is shown prior to insertion in a bone cavity 408. When the bone plug is inserted into the bone, the compression ridges are compressed radially as shown in FIG. 4B. The compression ridges exert outward pressure on the interior of the bone cavity. It will be recognized that the plug shown in FIG. 4 may have a hollow stem similar to the stem shown in FIG. 2A or a non-hollow stem similar to the stem shown in FIG. 1A. It will further be recognized that the plug shown in FIG. 4 may have a knob-like head similar to the head shown in FIG. 3A in lieu of the dome like head shown in FIG. 4.

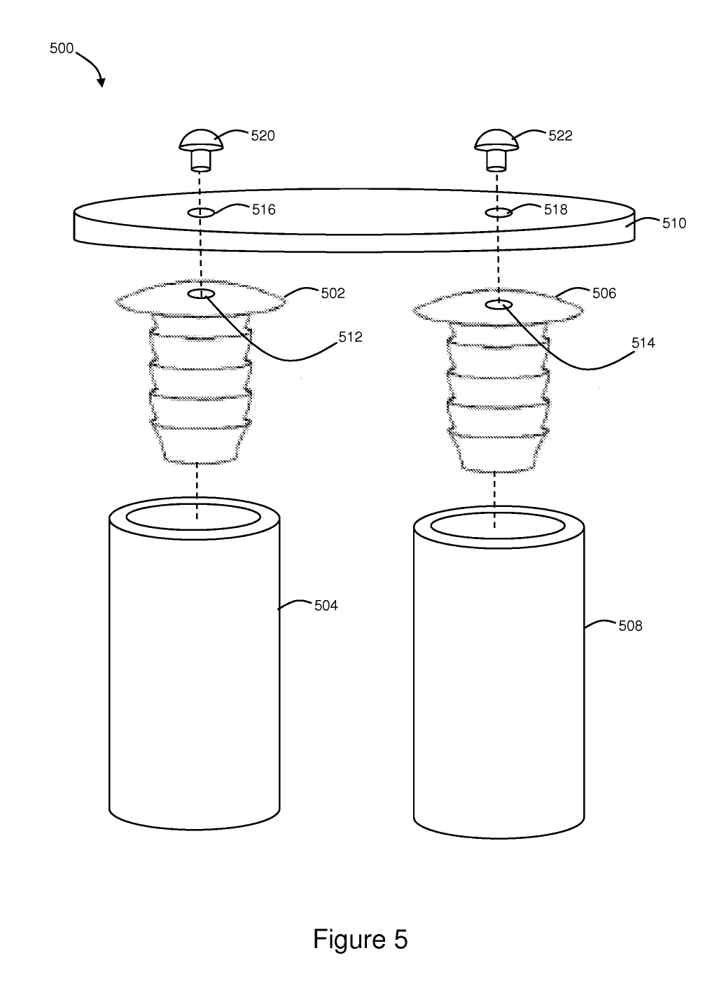

Referring now to FIG. 5, there is shown an illustrative bridging device 500 for bridging two bone plugs placed in parallel amputated bones. The bridging device may be used, for example, when the tibia and fibula are amputated. The first bone plug 502 is inserted into a first amputated bone 504, e.g., a tibia. The second bone plug 506 is inserted into a second amputated bone 508, e.g., a fibula. The first and second bone plugs may be any of the bone plug embodiments illustrated in FIGS. 1-4. The bone plugs each have a means to receive a fastening device, such as the sockets 512 and 514 shown in bone plugs 502 and 506, respectively. The bridge 510 also has means to receive a fastening device, such as holes 516 and 518 to receive fasteners 520 and 522, respectively. It will be realized that other fastening methods may be used to secure the bridge to the bone plugs. The bridge stabilizes the first bone relative to the second bone and distributes the weight applied to the amputated limb, relieving pressure on the tissue covering the amputated bone.

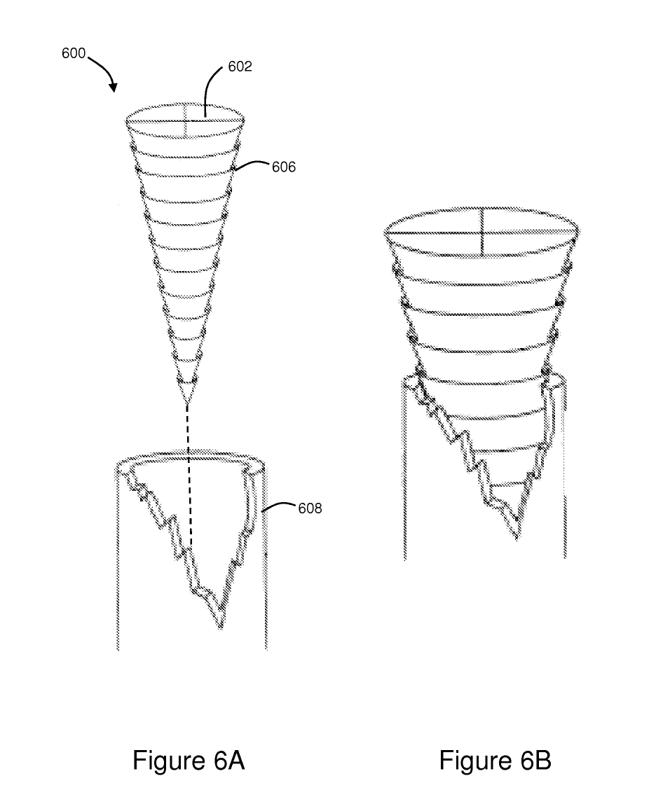

Referring to FIG. 6, there is shown an illustrative bone plug 600 according to a sixth embodiment. The bone plug 600 has a conical stem 602 having compression ridges 606 oriented around the circumference of the stem. The conical stem of the bone plug advantageously allows the plug to be adapted to different sized bones. The bone plug shown in FIG. 6 may thus be applied in a situation in which an emergency responder must prepare a patient having a severed limb or a limb requiring amputation for transportation to a place where an operation can be performed on the leg. Bone plug 600 aids in keeping the bone cavity free of foreign matter while the patient is being treated and transported. The bone plug may also aid in stabilizing a jagged edge of a bone, as demonstrated at 608, to prevent the jagged edge from harming surrounding tissue and potentially prevent further breakage of the bone. Bone plug 600 is shown inserted into bone cavity 608 in FIG. 6B.

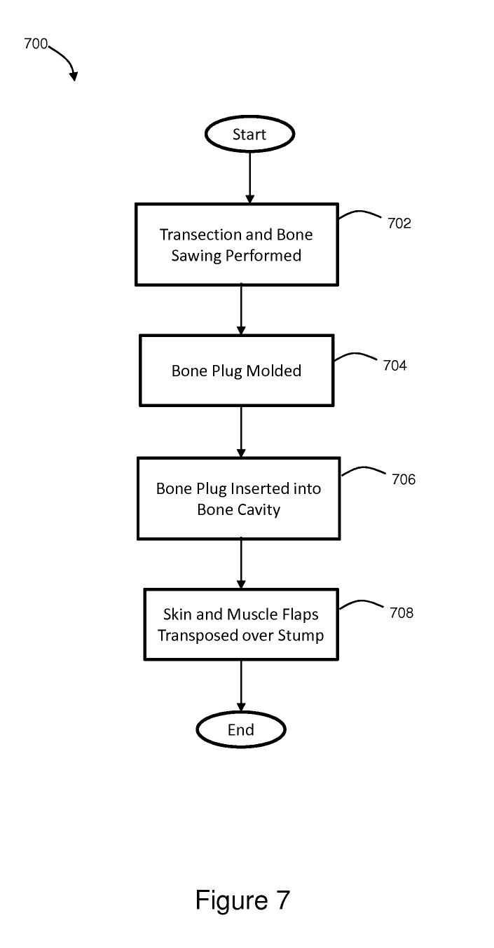

Referring now to FIG. 7, there is shown an illustrative flowchart 700 for inserting a bone plug into an amputated bone according to one embodiment of the invention. The method begins at block 702, at which the transection of the tissue and bone sawing is performed. If autodonated bone is to be used in the fabrication of the bone plug, the bone that has been removed from the patient is ground and used in the preparation of the bone matrix material. A bone plug is formed from the bone matrix material as indicated at block 704. The stem portion of the bone plug is sized to fit within the cavity of the amputated bone. If the bone plug is fabricated from synthetic matrix material or material from another donor, block 704 may precede block 702. The method proceeds to block 706, at which the bone plug is inserted into the bone cavity. Skin and muscle flaps preserved during the transaction are folded over the bone plug as indicated at block 708.

It is to be understood that the detailed description of illustrative embodiments are provided for illustrative purposes. The scope of the claims is not limited to these specific embodiments or examples. Therefore, various process limitations, elements, details, and uses can differ from those just described, or be expanded on or implemented using technologies not yet commercially viable, and yet still be within the inventive concepts of the present disclosure. The scope of the invention is determined by the following claims and their legal equivalents.

What is claimed is:

1. A device for insertion into an amputated bone, the device comprising:
a stem configured to be inserted into a cavity of the amputated bone;
a head coupled to the stem, the head having a diameter greater than the diameter of the stem, the head having a flat surface configured to interface with a severed surface of the amputated bone;
the head and stem fabricated from a modable porous matrix material configured to promote bone growth within the matrix.

2. The device of claim 1, further comprising compression ridges oriented around the circumference of the stem.

3. The device of claim 1, further comprising compression ridges oriented along the length of the stem.

4. The device of claim 1, wherein the stem comprises a shaft.

5. The device of claim 1, wherein the matrix material comprises material taken from bone of the amputee.

6. The device of claim 1, further comprising a tissue adhesive material affixed to the rounded surface of the head.

7. A device for bridging a first amputated bone and a second amputated bone, the device comprising:
a first stem configured to be inserted into a cavity of the first amputated bone;
a second stem configured to be inserted into a cavity of the second amputated bone;
a first head coupled to the first stem, the first head having a diameter greater than the diameter of the first stem, the first head having a flat surface configured to interface with a severed surface of the first amputated bone;
a second head coupled to the second stem, the second head having a diameter greater than the diameter of the second stem, the second head having a flat surface configured to interface with a severed surface of the second amputated bone;
a bridging plate coupled to the first head and the second head.

8. The device of claim 7, further comprising compression ridges oriented around the circumference of the first stem and the second stem.

9. The device of claim 7, further comprising compression ridges oriented along the length of the first stem and the second stem.

10. The device of claim 7, wherein the first stem and the second stem each comprise a shaft.

11. The device of claim 7, wherein the first head, first stem, second head and second stem are fabricated from a matrix material that comprises material taken from bone of the amputee.

12. The device of claim 7, further comprising a tissue-adhesive material affixed to the rounded surfaces of the first head and the second head.

15. A method for inserting a bone plug into an amputated bone, the method comprising:
molding a head and stem from a porous matrix material configured to promote bone growth in the matrix, such that:
the head has a diameter greater than the diameter of the stem;
the head has a flat surface configured to interface with a severed surface of the amputated bone;
the head is coupled to the stem; and
inserting the stem into a cavity of the amputated bone.

16. The method of claim 15, wherein the stem is molded having compression ridges oriented around the circumference of the stem.

17. The method of claim 15, wherein the stem is molded having compression ridges oriented along the length of the stem.

18. The device of claim 15, wherein the stem is molded such that it comprises a shaft.

19. The device of claim 15, wherein the head and stem are fabricated from a matrix material that comprises material taken from bone of the amputee.

20. The method of claim 15, further comprising affixing a tissue-adhesive material to the head.
